(19) United States
(12) Patent Application Publication
(10) Pub. No.: US 2003/0187349 A1
(43) Pub. Date: Oct. 2, 2003

(54) Sentinel lymph node detecting method

(75) Inventors: Mamoru Kaneko, Hanno-shi (JP); Toshihiro Shizuka, Tokyo (JP); Tadahiro Nakano, Tokyo (JP); Masaru Sudo, Tokyo (JP); Hisao Yabe, Tokyo (JP); Hitoshi Ueno, Tokyo (JP)

Correspondence Address: SCULLY SCOTT MURPHY & PRESSER, PC, 400 GARDEN CITY PLAZA, GARDEN CITY, NY, 11530

(73) Assignee: OLYMPUS OPTICAL CO., LTD., Tokyo (JP)

(21) Appl. No.: 10/396,034

(22) Filed: Mar. 25, 2003

(30) Foreign Application Priority Data
Mar. 29, 2002 (JP) 2002-097423
Mar. 29, 2002 (JP) 2002-097422

(51) Int. Cl. A61B 5/05

(52) U.S. Cl. 600/425

(57) ABSTRACT

A sentinel lymph node detecting system comprises an inserting portion with a small diameter which can be inserted into the body cavity, an X-ray detecting unit for two-dimensionally detecting X-rays, and an optical imaging unit 3 for taking visible-light images, which are mounted to the tip of the inserting portion. The X-ray detecting unit and the optical imaging unit are disposed along the longitudinal direction of the inserting portion, and are closely disposed one to another so as to observe in the same direction. A radioactive tracer accumulated in sentinel lymph nodes is detected by the X-ray detecting unit. The X-ray detected image is smaller in size than the image taken by the optical imaging unit. Thus, sentinel lymph nodes can be quickly identified in a sure manner, and further, the load placed on a patient, such as the need to perform laparotomy, can be reduced.

This application claims benefit of Japanese Applications No. 2002-97423 filed in Japan on Mar. 29, 2002 and No. 2002-97422 filed in Japan on Mar. 29, 2002, the contents of which are incorporated by this reference.

### BACKGROUND OF THE INVENTION

1. Field of the Invention

The present invention relates to a sentinel lymph node detecting method for identifying sentinel lymph nodes near affected portions in the body cavity.

2. Description of the Related Art

Presently, examination for the presence or absence of metastasis or scattering of tumors is a primary decision factor for effective treatment for a patient with cancer. Tumor cells which spread from the primary origin of the tumor, which is an affected portion, to lymphatic vessels first reach sentinel lymph nodes. In the early stage of cancer, it is known that the probability of metastasis to lymph nodes is approximately 20%, and metastasis does not occur in the remaining 80%. In the event that metastasis does not occur, a complete recovery can be expected by excision within the range of the primary origin of the tumor. The sentinel lymph nodes are in the lower portion of the affected portion which is on the surface of the body cavity such as the stomach, large intestine, or the like, so the sentinel lymph nodes cannot be directly observed by external observation. Accordingly, examination is required wherein the sentinel lymph nodes near the affected portion are indirectly detected and accurately identified, and the cells of the sentinel lymph nodes are sampled and sent to a laboratory.

Now, as a reliable system for identifying the sentinel lymph nodes, the sentinel lymph node detecting method disclosed in Japanese Unexamined Patent Application Publication No. 2001-299676 is known. In this detecting method, indocyanine-green, which is an infrared fluorescent dye, is locally injected around the tumor as a tracer. Following a predetermined time period, laparotomy is performed, and near-infrared excitation light is cast on the portion to be observed. Near-infrared fluorescence is generated from the sentinel lymph nodes due to accumulation of the indocyanine-green. The near-infrared fluorescence is converted into visible light and is observed as a visible image, so that the sentinel lymph nodes can be detected.

However, with the sentinel lymph node detecting method disclosed in the aforementioned Japanese Unexamined Patent Application Publication No. 2001-299676, there is the problem that laparotomy is required at the time of the examination.

Also, a system for specifying the position of sentinel lymph nodes, disclosed in Japanese Unexamined Patent Application Publication No. 9-189770, is known. The system specifies the position of the sentinel lymph nodes by detecting photon emission sources, and more specifically, a radioactive substance in the lymphatic vessels is traced by means of a conventional hand-held probe under laparotomy, and the position of the sentinel lymph nodes containing the radioactive substance at high density therein is specified. Note that the above-described probe is a gamma-ray probe which employs crystals of cadmium telluride as a photon-radiation detecting sensor.

However, with the system for specifying the position of sentinel lymph nodes disclosed in Japanese Unexamined Patent Application Publication No. 9-189770, the conventional probe is employed, so laparotomy is required for the examination, and giving consideration to the QOL (quality of life) of the patient, the burden placed on the patient is great. Also, even if the sentinel lymph nodes are detected, confirmation of the spread of cancer is required following the detection. This means that a surgeon might lose sight of the position of the sentinel lymph nodes in the step of sampling tissue from the portion. Also, the sentinel lymph nodes are identified as points, leading to problems of taking a long time for detection.

### SUMMARY OF THE INVENTION

Accordingly, it is an object of the present invention to solve the above problems and provide a sentinel lymph node detecting method wherein sentinel lymph nodes can be quickly identified in a sure manner, and the load placed on a patient, such as the need to perform laparotomy, can be reduced.

According to a first aspect of the present invention, a sentinel lymph node detecting method uses a sentinel lymph node observation system which comprises: an inserting portion with a small diameter which can be inserted into the body cavity; an X-ray detector at the tip of the inserting portion for two-dimensionally detecting X-rays; and an optical imaging device for taking visible-light images; wherein the X-ray detector and the optical imaging device are positioned at the inserting portion in the longitudinal direction thereof, and are closely positioned one to another so as to observe in the same direction; and wherein sentinel lymph nodes in which a radioactive tracer has been accumulated are detected by the X-ray detector.

According to another aspect of the present invention, a sentinel lymph node detecting method uses: an X-ray source and an X-ray camera, for observing sentinel lymph nodes near affected portions in which an imaging agent for absorbing X-rays has been accumulated beforehand; an endoscope for observing the body cavity; and a marking means inserted from a forceps opening of the endoscope; wherein marking is made with the marking means onto the inner wall of the body cavity organ near the portion in which the imaging agent has been accumulated.

Other features and advantages of the present invention will become apparent from the following description.

### BRIEF DESCRIPTION OF THE DRAWINGS

FIG. 1 is a diagram which illustrates the configuration of a sentinel lymph node observation system to which a sentinel lymph node detecting method according to a first embodiment of the present invention has been applied;

FIG. 2 illustrates a display screen on an image display unit of the sentinel lymph node observation system shown in FIG. 1;

FIG. 3 is a cross-sectional view which illustrates the detailed configuration of an X-ray camera unit of the sentinel lymph node observation system shown in FIG. 1;

FIG. 4 is a cross-sectional view which illustrates a modification of an X-ray camera of the sentinel lymph node observation system shown in FIG. 1;

FIG. 5 is a diagram which illustrates the configuration of a sentinel lymph node observation system according to a second embodiment of the present invention;

FIG. 6 is a cross-sectional view which illustrates the configuration of a scintillator probe unit for application to the sentinel lymph node observation system according to the second embodiment shown in FIG. 5;

FIG. 7 is a view taken along the arrow B in FIG. 6;

FIG. 8 is a cross-sectional view of a first modification of the scintillation probe for application to the sentinel lymph node observation system according to the second embodiment shown in FIG. 5;

FIG. 9 is a view taken along the arrow C in FIG. 8;

FIG. 10 is a cross-sectional view which illustrates the tip of a scintillation probe of a second modification corresponding to the scintillation probe for application to the sentinel lymph node observation system according to the second embodiment shown in FIG. 5;

FIG. 11 is a cross-sectional view which illustrates the tip of a scintillation probe of a third modification corresponding to the scintillation probe for application to the sentinel lymph node observation system according to the second embodiment shown in FIG. 5;

FIG. 12 is a cross-sectional view which illustrates the tip of a scintillation probe of a fourth modification corresponding to the scintillation probe for application to the sentinel lymph node observation system according to the second embodiment shown in FIG. 5;

FIG. 13 is a diagram which illustrates the configuration of a sentinel lymph node observation system according to a third embodiment of the present invention, and a cross-section of an X-ray detecting unit making up the system;

FIG. 14 is a cross-sectional view which illustrates a modification of a puncture needle for application to the sentinel lymph node observation system according to the third embodiment shown in FIG. 13;

FIG. 15 is a perspective view which illustrates the tip of an inserting portion of an endoscope of a sentinel lymph node observation system according to a forth embodiment of the present invention;

FIG. 16 is a block configuration diagram which illustrates an ultrasonic transducer/X-ray detector control unit of the sentinel lymph node observation system according to the fourth embodiment shown in FIG. 15;

FIG. 17 illustrates a display screen of an ultrasonic/X-ray image while observing an SN position using the sentinel lymph node observation system according to the fourth embodiment shown in FIG. 15;

FIG. 18 is a side view which illustrates a modification of an inserting portion of an endoscope of the sentinel lymph node observation system according to the fourth embodiment shown in FIG. 15;

FIG. 19 is a diagram which illustrates a display screen on an image display unit in the event of applying the inserting portion of the endoscope of the modification shown in FIG. 18;

FIG. 20 is a cross-sectional view which illustrates an inserting portion of an endoscope of a sentinel lymph node observation system to which a sentinel lymph node detecting method according to a fifth embodiment of the present invention has been applied;

FIG. 21 is a cross-sectional view which illustrates an inserting portion of an endoscope of a sentinel lymph node observation system to which a sentinel lymph node detecting method according to a sixth embodiment of the present invention has been applied;

FIG. 22 is a diagram which illustrates the configuration of a sentinel lymph node observation system to which a sentinel lymph node detecting method according to a seventh embodiment of the present invention has been applied;

FIG. 23A is a diagram which illustrates a display screen on an X-ray image display unit of the sentinel lymph node observation system according to the seventh embodiment shown in FIG. 22;

FIG. 23B is a diagram which illustrates a display screen on an endoscope image display unit of the sentinel lymph node observation system according to the seventh embodiment shown in FIG. 22;

FIG. 24 is a diagram which illustrates the configuration of a sentinel lymph node observation system to which a sentinel lymph node detecting method according to an eighth embodiment of the present invention has been applied;

FIG. 25A is a diagram which illustrates a display screen on an X-ray image display unit of the sentinel lymph node observation system according to the eighth embodiment shown in FIG. 24;

FIG. 25B is a diagram which illustrates a display screen on an ultrasonic image display unit of the sentinel lymph node observation system according to the eighth embodiment shown in FIG. 24;

FIG. 26 is a diagram which illustrates the configuration of a sentinel lymph node observation system to which a sentinel lymph node detecting method according to a ninth embodiment of the present invention has been applied;

FIG. 27 is a diagram which illustrates the configuration of a sentinel lymph node observation system to which a sentinel lymph node detecting method according to a tenth embodiment of the present invention has been applied;

FIG. 28 is a diagram which illustrates the configuration of a sentinel lymph node observation system to which a sentinel lymph node detecting method according to an eleventh embodiment of the present invention has been applied.

### DETAILED DESCRIPTION OF THE PREFERRED EMBODIMENTS

Embodiments according to the present invention will be described below with reference to the drawings.

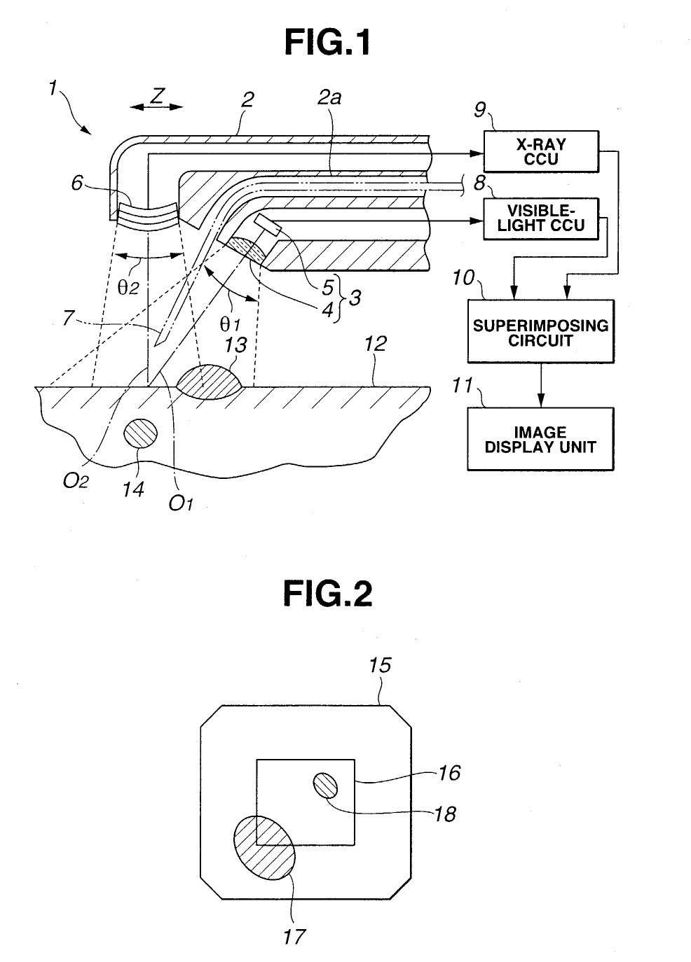

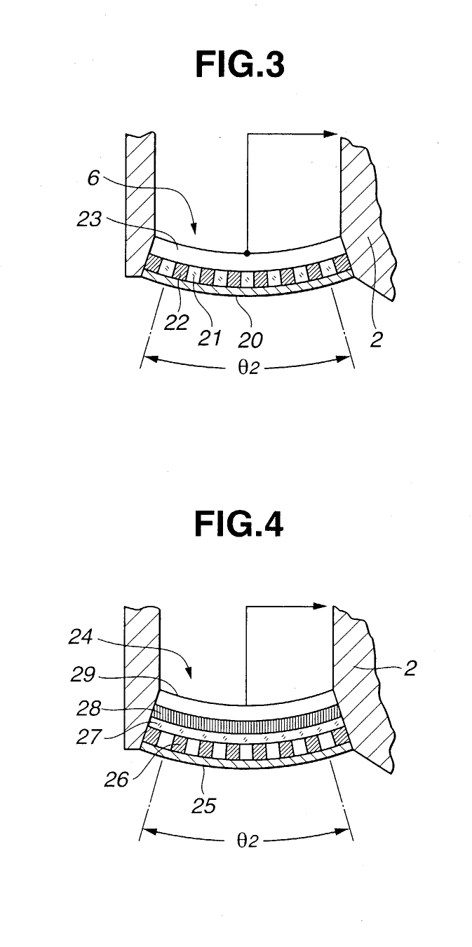

FIG. 1 is a diagram which illustrates the configuration of a sentinel lymph node observation system to which a sentinel lymph node detecting method as a first embodiment of the present invention has been applied. FIG. 2 illustrates a display screen of an image display unit of the system. FIG. 3 is a cross-sectional view which illustrates the configuration of an X-ray camera unit according to the system in detail.

A sentinel lymph node observation system 1 of the present embodiment is a system for detecting radioactive substances (tracers), which flow into a sentinel lymph node (which will be referred to as “SN”) 14 positioned underneath an affected portion 13 on the wall of a body cavity 12 of an internal organ such as the stomach, by means of an X-ray camera, and identifying the SN.

The above-described sentinel lymph observation system 1 generally comprises a flexible endoscope (not shown) having an inserting portion 2 with a small diameter capable of being inserted into the body cavity, a visible-light CCU (camera control unit) 8 for a visible-light camera, an X-ray CCU (camera control unit) 9 for an X-ray camera, a superimposing circuit 10 for superimposing an output image from the X-ray CCU 9 on an output image from the visible-light CCU 8, and an image display unit 11 for displaying an output image from the superimposing circuit 10.

A visible-light camera unit 3 and an X-ray camera unit 6 are disposed at the tip of the inserting portion 2, which are close one to another along the longitudinal direction Z of the inserting portion. The visible-light camera unit 3 and the X-ray camera unit 6 are camera units which can take visible-light images and X-ray images of the body cavity alongside the longitudinal direction Z. Furthermore, a marker injection needle 7 or an aspiration biopsy needle can be slidably inserted into a forceps opening 2a so as to protrude at the position between the visible-light camera unit 3 and the X-ray camera unit 6 at the tip of the inserting portion 2.

The visible-light camera 3 comprises an object lens 4 and a CCD 5 which is an optical imaging device, has an imaging optic axis O1 inclined at a predetermined angle with regard to the inserting unit longitudinal direction Z, and has an imaging angular field of view θ1.

The X-ray camera unit 6 consists of an X-ray detecting unit having the directivity of a predetermined angular range. The X-ray detecting unit comprises a curved convex shield 20 for shielding visible light and passing X-rays, a scintillator 21 which is an X-ray detecting device, a collimator 22 which is a collimating means, and a curved CCD 23 disposed along the inner face of the scintillator 21, for converting a visible-light image converted at each element of the scintillator 21 into electric signals.

The collimator 22 is made up of a material which can block X-rays (radial rays), such as lead or flint glass. Moreover, the collimator 22 has a great number of apertures each of which have an element of the scintillator 21 so as to be surrounded thereby.

The scintillator 21 is an X-ray detecting device consisting of multiple minute elements which are disposed in a convex manner along the inside face of the shield 20 when two-dimensionally viewed. Each element of the scintillator 21 generates light-photons from X-rays which have reached the element.

Each element of the scintillator 21 is disposed in an aperture of the collimator 22 in a curved shape when two-dimensionally viewed, for yielding directivity. The array pitch of the apertures of the collimator 22 defines the spatial resolution of the camera. On the other hand, the scintillator 21 of which elements are disposed in a curved shape when two-dimensionally viewed defines an X-ray imaging angular field of view θ2 of the X-ray camera unit 6, when two-dimensionally viewed. Let us say that the X-ray imaging angular field of view θ2 is smaller than the angular field of view θ1 of the visible-light camera unit 3. Here, the range in which the X-ray camera unit 6 can take images is less than that in which the visible-camera 3 can take images. Moreover, the X-ray imaging axis O2 which is a center axis of the X-ray imaging angular field of view θ2 of the X-ray camera unit 6 is generally orthogonal to the inserting portion longitudinal direction Z. Furthermore, the X-ray imaging axis O2 and the imaging axis O1 of the visible camera unit 3 intersect, or the X-ray imaging axis O2 passes through near the imaging axis O1.

The X-ray camera unit 6 detects the intensity of the X-rays from an RI colloid of a radioactive substance, accumulated in the affected portion or the SN, and the X-ray output is acquired by the X-ray camera CCU 9.

Now, description will be made with regard to displaying images on the image display unit 11, taken by the visible-light camera unit 3 and the X-ray camera unit 6. As shown in FIG. 2, an image which has been taken by the visible-light camera unit 3 is displayed on the entire area of a visible-light image displaying screen frame 15. Furthermore, a two-dimensional X-ray image acquired from the X-ray camera unit 6 is displayed on an X-ray image displaying screen frame 16 which is smaller than the above-described display screen frame 15, in a superimposed manner.

The injection needle 7 is a needle for injecting an RI colloid agent as a tracer around the affected portion 13 on the wall of the body cavity.

Now, description will be made with regard to a case of identifying the SN position by means of the sentinel lymph node observation system 1 according to the present invention having the configuration as described above. First of all, a user inserts the injection needle 7 into the corner of the affected portion 13 on the wall of the body cavity 12, and injects an RI colloid agent as an SN detecting tracer while observing the visible-light image acquired from the visible-light camera unit 3 and displayed on the image display unit 11. Note that an RI colloid agent such as Tc-Sn colloid, for example, is employed, giving consideration to the nature of inflow for SNs and the nature of accumulation.

Following a predetermined time period, the RI colloid reaches the SN portion. At that time, the image of the area around the affected portion 13 acquired from the visible-light camera unit 3 is displayed on the displaying screen frame 15 of the image display unit 11. At the same time, the X-ray image acquired from the X-ray camera unit 6 is displayed on the displaying screen frame 16 within the above-described displaying screen frame 15 in a superimposed manner. In the event that the SN 14 is within the imaging angular field of view θ2 of the X-ray camera unit 6, the X-rays emitted from the RI colloid agent accumulated in the SN 14 are detected, and are displayed on the displaying screen frame 16. The SN image 18 acquired from the X-ray camera unit 6 is superimposed on the image acquired from the visible-light camera unit 3, and is displayed on the displaying screen frame 16 as shown in FIG. 2, so that the SN position is detected (identified). Other SNs are identified while moving the inserting portion 2 around the affected portion 13 and observing the X-ray image displayed on the displaying screen frame 16.

Also, a tissue sample can be taken by marking the detected SN position with the injection needle 7, or inserting the aspiration biopsy needle into the forceps opening 2a of the inserting portion 2, and then inserting the injection needle into the SN 14 while observing the image displayed on the displaying screens 15 and 16.

With the sentinel lymph node observation system 1 according to the embodiment as described above, while the visible-light images and the X-ray images of the body cavity in a predetermined direction of the inserting portion 2 are simultaneously taken by the visible-light camera unit 3 and the X-ray camera unit 6, the X-ray image is superimposed on the visible-light image, so that the SN positions can be easily identified. In the case of the present embodiment, in particular, the collimator means is disposed at the X-ray input area of the X-ray camera 6, so the directivity of the scintillator disposed two-dimensionally is expanded, and thus X-ray images can be taken over a wide range. Accordingly, the SN can be efficiently detected over a even wider range.

Also, the portions of the X-ray image which is to be superimposed on the display screen of the image display unit 11 wherein the X-ray intensity is high, that is to say the SN portions, are displayed as a computer color-enhanced image, so the SN positions can be detected more easily.

Next, a modification with regard to the configuration of the X-ray camera 6 applied to the sentinel lymph node observation system 1 of the first embodiment as described above will be described with reference to a cross-sectional view illustrating the X-ray camera of the modification shown in FIG. 4.

An X-ray camera unit 24 of the present modification consists of an X-ray detecting unit having directivity of expanded imaging angular field of view, the same as the above-described X-ray camera unit 6. The present modification is different from the first embodiment as described above in that a curved convex scintillator sheet and an optical fiber sheet is mounted instead of multiple scintillator elements 21 applied to the X-ray camera unit 6. That is to say, the X-ray camera unit 24 comprises a curved convex shield 25 for shielding visible light and so forth and passing X-rays, a collimator 26 which is a collimator means disposed at the inside of the shield 25, a scintillator sheet 27 which is an X-ray detecting device disposed at the inside of the collimator 26, an optical fiber sheet 28 disposed at the inside of the scintillator sheet 27, and a CCD 29 disposed at the inside of the optical fiber sheet 28 as shown in FIG. 4.

The collimator 26 is disposed along the inside face of the shield 25, and is made up of an X-ray shielding material having multiple apertures for yielding the directivity of incident X-rays.

The scintillator sheet 27 is a curved convex X-ray detecting device expanding two-dimensionally along the inside of the collimator 26, and the scintillator sheet 27 converts X-rays into photons.

The optical fiber sheet 28 is a curved convex fiber sheet disposed along the inside of the scintillator sheet 27, and allows transmission of only visible light which has been converted from X-rays by the scintillator sheet 27, input in the direction of the directivity thereof.

The CCD 29 is a curved convex CCD disposed along the inside of the scintillator sheet 27, and converts the visible light from each fiber unit, which has been passed through the optical fiber sheet 28, into electric signals as X-ray image signals.

The X-ray camera unit 24 has an X-ray imaging angular field of view θ2 expanding two-dimensionally, the same as in the case of the above-described X-ray camera unit 6. The X-ray imaging angular field of view θ2 is smaller than the imaging angular field of view θ1 of the visible-light camera unit 3. Also, only the light incoming along the direction of the directivity, which is visible light converted from X-rays by the scintillator sheet 27, passes through the optical fiber sheet 28, reaches the CCD sheet 29, and is acquired as X-ray image signals. Note that the X-rays which have not been converted into visible light and have not been cast along the direction of the directivity do not pass through the optical fiber sheet 28, and accordingly the CCD sheet 29 is protected from X-rays.

With the X-ray camera unit 24 according to the present modification, a film-shaped scintillator sheet 27 can be employed instead of a great number of scintillator elements, thereby facilitating manufacturing of the X-ray camera unit, and thus a low-cost X-ray camera can be provided.

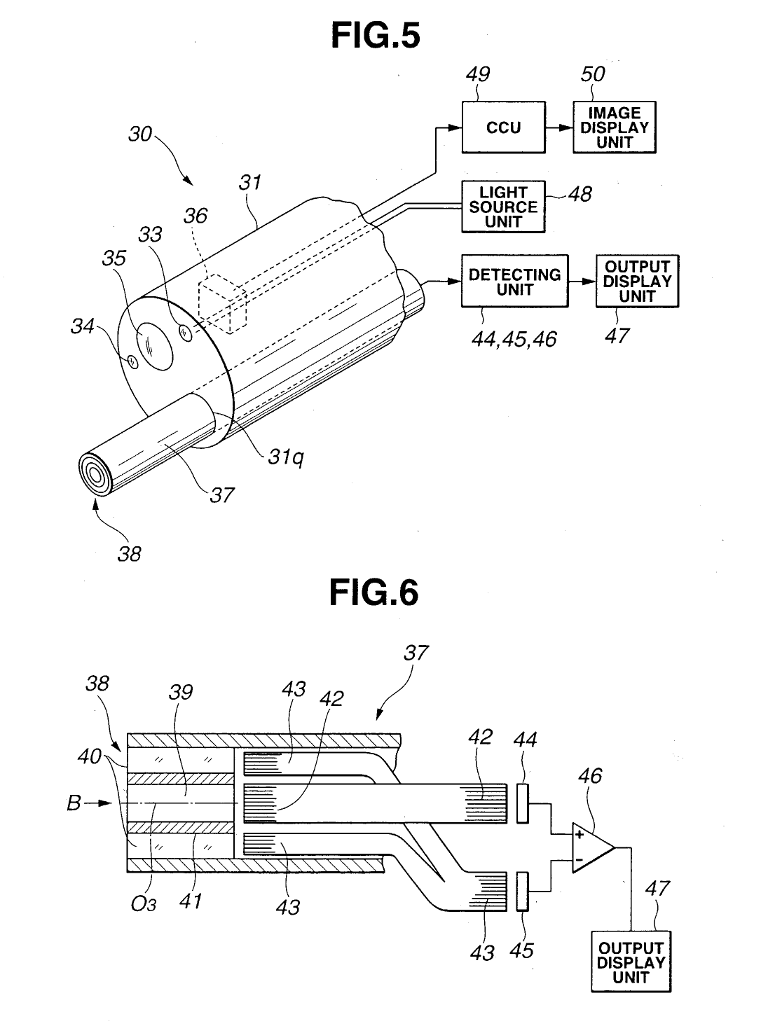

Next, a sentinel lymph node observation system to which a sentinel lymph node detecting method according to a second embodiment of the present invention has been applied, will be described with reference to FIGS. 5 through 7.

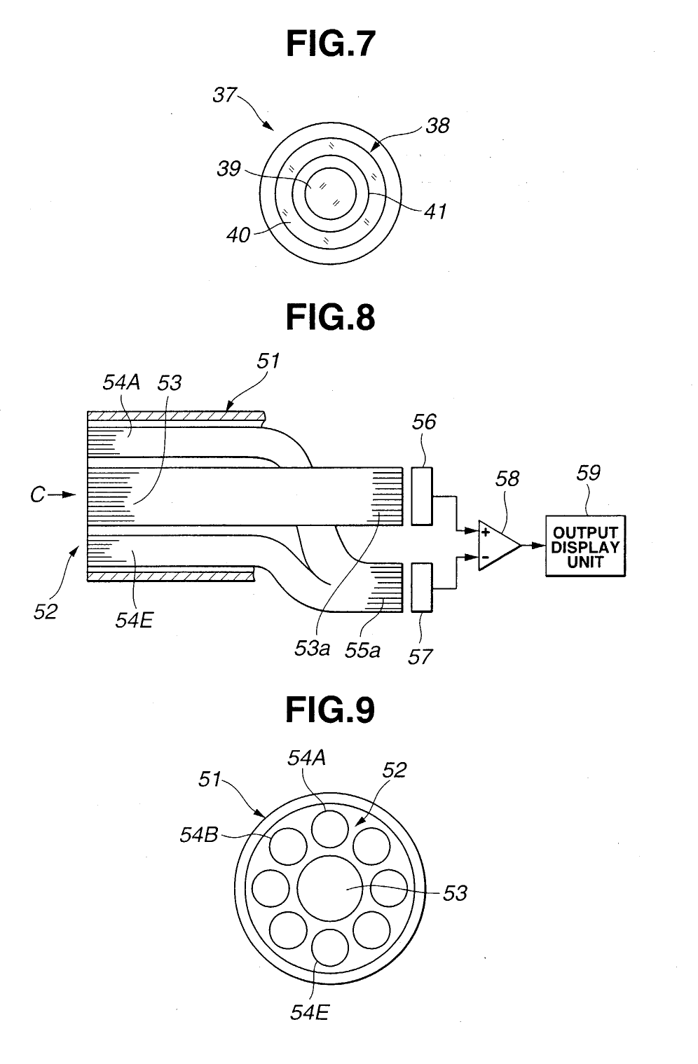

FIG. 5 is a diagram which illustrates the configuration of the sentinel lymph node observation system according to the present embodiment, and FIG. 6 is a cross-sectional view which illustrates the configuration of the scintillator probe unit employed in the sentinel lymph node observation system. FIG. 7 is a view taken along the arrow B in FIG. 6.

As shown in FIG. 5, a sentinel lymph node observation system 30 of the present embodiment generally comprises an endoscope (not shown) having an inserting portion 31 which is capable of being inserted into the body cavity, a lighting unit 48 for lighting the body cavity, a CCU 49 built in the inserting portion 31 for controlling a visible-light camera CCD 36, an image display unit 50 for acquiring image information from the CCU 49 and displaying visible-light images, a scintillation probe 37 which is an inserting portion with a small diameter, a scintillator output detecting unit which will be described later, and a scintillator output display unit 47.

Illumination lenses 33 and 34, an objective lens 35, and a visible-light CCD 36, are disposed at the tip of the inserting portion 31, and furthermore, the scintillation probe 37 is inserted into a forceps opening 31a of the inserting portion 31.

The scintillator output detecting unit comprises two photoelectric multiplier tubes PMT(A) 44 and PMT(B) 45, and a differential motion amplifier 46 which is a detecting means to which the outputs of the PMT(A) 44 and the PMT(B) 45 are input.

The scintillation probe 37 includes an X-ray detecting unit 38 at the tip thereof, which comprises a center scintillator unit 39 as a minute rod-shaped inner detecting unit having an axis parallel to the probe longitudinal direction Z, and a peripheral scintillator unit 40 as a minute cylinder-shaped outer detecting unit positioned at the outside of the center scintillator 39. Furthermore, an X-ray shielding wall portion 41 is formed between the scintillator units 39 and 40. Moreover, optical fibers 42 and 43, serving as light-input units, are disposed at the back of the center scintillator 39 and the peripheral scintillator 40, respectively, so as to face one to another. The X-ray detecting axis O3 of the X-ray detecting unit 38 is parallel with the Z direction, and has directivity parallel thereto.

The optical fibers 42 and 43 are extended into the tip of the base portion of the endoscope, and the light-output units of the optical fibers 42 and 43 are positioned so as to face to the PMT(A) 44 and the PMT(B) 45, respectively, positioned at the tip of the base.

With the scintillation probe 37 as described above, X-rays incident to the center scintillator unit 39 along the Z direction are converted into visible light in the interior of the scintillator, the visible light is input to the receiving face of the PMT(A) 44 via the optical fiber 42, and the amount of light is detected. On the other hand, X-rays incident to the peripheral scintillator unit 40 along the Z direction are converted into visible light in the interior of the scintillator, the visible light is input to the receiving face of the above-described PMT(B) 45 via the optical fiber 43, and the amount of light is detected.

The detected output from the PMT(A) 44 and PMT(B) 45 is input to the positive terminal or the negative terminal of the differential motion amplifier 46. In the event that the X-ray intensities input to the center scintillator and the peripheral scintillator are different one from another, the output of the differential motion amplifier 46 changes to the positive or the negative. The differential output from the differential motion amplifier 46 is output to the output display unit 47, and the output value is displayed. The user can judge whether the X-ray density of the center scintillator unit 39 or the peripheral scintillator unit 40 is greater, from the displayed value.

Now, the method for identifying the SN position by the sentinel lymph node observation system 30 according to the present embodiment having the above-described configuration will be described.

In the case of the present system, an RI colloid serving as a radioactive substance tracer is also injected around the affected portion on the body cavity, and the RI colloid is accumulated in the SN corresponding to the affected portion. In this state, the inserting portion 31 is inserted into the body cavity, and the tip of the scintillation probe 37 is positioned near the affected portion so as to face one to another while observing the images displayed on the image display unit 50 from the visible-light camera. Subsequently, X-rays emitted from the RI colloid accumulated in the SN are detected by the tip of the scintillation probe 37, and the intensity is displayed on the output display unit 47 to be judged.

In the event that a positive value is displayed on the output display unit 47, judgment is made that the SN is positioned in front of the center scintillator unit 39. Conversely, in the event that a negative value is displayed on the output display unit 47, judgment is made that the SN is not positioned in front of the center scintillator unit 39, but is positioned in front of the peripheral scintillator unit 40, that is to say, is positioned offset from the front of the center scintillator unit 39. In the event that zero or an undetermined value is displayed, judgment is made that the SN is not positioned near the front of the scintillation probe 37.

A state wherein the SN is positioned in front of the center scintillator unit 39 is searched for, by moving the scintillation probe 37 in the body cavity and observing the images on the output display unit 47, and the SN position is identified. In the event that the SN position is identified, the state of metastasis can be confirmed by marking the position to take a tissue sample later, or directly inserting a puncture needle to take a tissue sample.

With the sentinel lymph node observation system 30 according to the present embodiment, simply monitoring the output display unit 47 while moving the tip of the scintillation probe 37 in the body cavity enables accurate identification of the SN position by simple procedures.

Now, a scintillation probe of a first modification according to the scintillation probe 37 applied to the sentinel lymph node observation system 30 according to the above-described second embodiment will be described with reference to FIGS. 8 and 9.

FIG. 8 is a cross-sectional view illustrating the scintillation probe of the modification, and FIG. 9 is a view taken along the arrow C in FIG. 8.

The scintillation probe 51 of the present modification includes an X-ray detecting unit 52 which comprises a center scintillation fiber 53 stored in the probe, and multiple peripheral scintillation fibers 54A, 54B, . . . , 54E, . . . , disposed around the center scintillation fiber 53 as shown in FIGS. 8 and 9. The scintillation fibers 53 and 54A, 54B, . . . , 54E, . . . , are extended to the base of the endoscope. The light-output unit 53a of the center scintillation fiber 53 is positioned so as to face a PMT(A) 56, which is a photoelectric multiplier tube, at the base portion. The peripheral scintillation fibers 54A, 54B, . . . , 54E, . . . , are bundled into one, and a light-output unit 55a of the bundled fiber is positioned so as to face a PMT(B) 57, which is a photoelectric multiplier tube.

The detected outputs from the PMT(A) 56 and the PMT(B) 57 are input to the positive terminal of the differential motion amplifier 58 and the negative terminal, respectively. The differential output from the differential motion amplifier 58 is output to the output display unit 59, and the output value is displayed thereon.

With the above-described scintillation probe 51, X-rays cast into the center scintillation fiber 53 are converted into visible light in the interior of the scintillation fiber, and the quantity of light is detected by the PMT(A) 56, the same as the above-described scintillation probe 37. On the other hand, X-rays cast into the peripheral scintillation fibers 54A, 54B, . . . , 54E, . . . , are converted into visible light in the interior of the scintillation fiber, and the quantity of light is detected by the PMT(B) 57. In the event that the X-ray intensities input to the center scintillation fiber 53 and the peripheral scintillation fibers 54A, 54B, . . . , 54E, . . . , are different one from another, the output of the differential motion amplifier 58 changes to the positive or negative, and the value is displayed on the output display unit 59. The user can judge whether X-ray intensity of the center scintillation fiber or the peripheral scintillation fibers is greater, from the displayed value.

The method for identifying the SN position by the sentinel lymph node observation system employing the scintillation probe 51 of the present modification having the above-described configuration is the same as the sentinel lymph node observation system 30 according to the above-described second embodiment.

With the scintillation probe 51 according to the present modification, scintillation fibers are employed instead of scintillators and optical fibers, and thus the number of components can be reduced, thereby enabling providing of a scintillation probe having a simple configuration, requiring no X-ray shield portion.

Note that with the scintillation probe 51 according to the above-described modification, an arrangement, wherein the light-output units of the peripheral scintillation fibers 54A, 54B, . . . , 54E, . . . , are not bundled into one, but rather are bundled into two or four, and each quantity of light is detected by a separate PMT, and the output from each PMT is input to the differential amplifier so as to detect the difference between the quantities of light from the center scintillation fiber 53 and each bundled peripheral scintillation fiber, enables detecting of the direction of the SN position in the event that the SN is at a near position other than the center position.

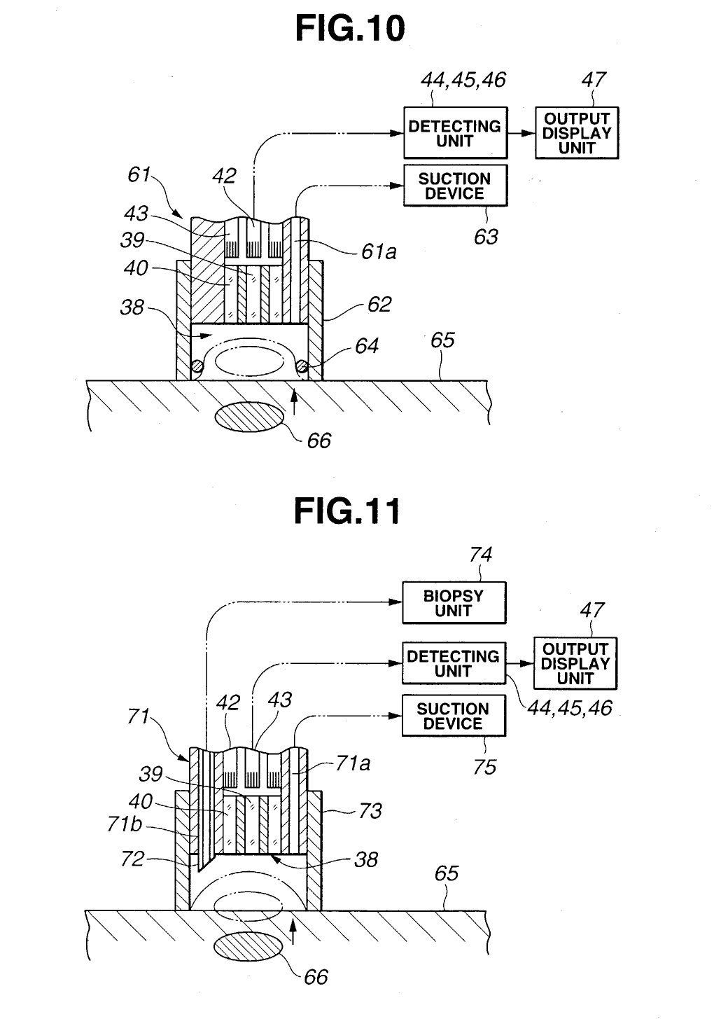

Now, a scintillation probe of a second modification corresponding to the scintillation probe 37 applied to the sentinel lymph node observation system 30 according to the above-described second modification will be described with reference to FIG. 10.

FIG. 10 is a cross-sectional view illustrating the tip of a scintillation probe according to the present embodiment.

As shown in FIG. 10, a suction passage 61a is provided to a scintillation probe 61 of the present modification as well as the X-ray detecting unit 38, and furthermore, an opening cap 62 holding a detachable rubber ring 64 within the inner circumference thereof is mounted to the tip of the probe. Moreover, a scintillator output detecting unit for the X-ray detecting unit 38, which will be described later, and a suction device 63 which is connected to the suction passage 61a, are disposed at the base of the scintillation probe 61.

The X-ray detecting unit 38 has the same configuration as that included in the scintillation probe 37 according to the above-described second embodiment.

The scintillator output detecting unit also has the same configuration as that included in the scintillation probe 37 of the observation system according to the above-described second embodiment, and comprises the PMT(A) 44 and the PMT(B) 45, which are photoelectric multiplier tubes, and the differential motion amplifier 46 to which the outputs of the PMT(A) 44 and the PMT(B) 45 are input. The differential motion output from the differential motion amplifier 46 is input to the output display unit 47, and the state of X-rays cast on the X-ray detecting unit 38 is displayed, the same as the above-described scintillation probe 37.

Using the scintillation, probe 61 of the present modification, the SN 66 underneath the wall of the body cavity 65 can be detected with the same method as the above-described first embodiment. Following detecting of the SN 66, the opening cap 62 at the tip of the probe 61 is pressed into contact against the wall underneath which the SN 66 is positioned. Subsequently, the SN 66 is sucked upward along with the wall portion of the body cavity 65 by suctioning air using suction device 63 as shown FIG. 10. The rubber ring 64 is snapped onto the wall of the cavity body 65 which is bulged, containing the SN 66 therein by the sucking action, and thus the SN position is marked. Subsequently, a surgeon inserts an aspiration biopsy needle into the SN 66 onto which the rubber ring is snapped so that a tissue sample of the SN 66 can be taken.

In the event that the scintillation probe 61 according to the present modification is used, marking of the SN 66 can be easily made.

Now, a scintillation probe of a third modification corresponding to the scintillation probe 37 applied to the sentinel lymph node observation system 30 according to the above-described second embodiment will be described with reference to FIG. 11.

FIG. 11 is a cross-sectional view illustrating the tip of a scintillation probe of the present modification.

As shown in FIG. 11, a suction passage 71a and a needle inserting opening 71b for inserting an aspiration biopsy needle 72 are provided to a scintillation probe 71 as well as the X-ray detecting unit 38, and furthermore, a opening cap 73 is mounted to the tip of the probe. Moreover, the scintillator output detecting unit for the X-ray detecting unit 38 and a suction device 75 for being connected to the suction passage 71a are provided to the base of the scintillation probe 71.

The X-ray detecting unit 38 has the same configuration as that included in the scintillation probe 37 according to the above-described second embodiment.

The scintillator output detecting unit also has the same configuration as that included in the scintillation probe 37 of the observation system according to the above-described second embodiment, and comprises the PMT(A) 44 and the PMT(B) 45, which are two photoelectric multiplier tubes, and the differential motion amplifier 46 to which the outputs from the PMT(A) 44 and the PMT(B) 45 are input. The differential motion output of the differential motion amplifier 46 is output to the output display unit 47, and the state of X-rays casting on the X-ray detecting unit 38 is displayed thereon, the same as the above-described scintillation probe 37.

Using the scintillation probe 71 of the present modification, the SN 66 underneath the wall of the body cavity 65 can be detected with the same method as the above-described first embodiment. Following the detecting, the opening cap 73 at the tip of the probe 71 is pressed into contact against the wall underneath which the detected SN 66 is positioned. Subsequently, the SN 66 is sucked upward along with the wall of the body cavity 65 by suctioning using the suction device 75 as shown in FIG. 11. The tissue sample of the SN 66 can be taken by inserting the aspiration biopsy needle 72 into the SN 66 while being sucked due to suction.

By using the scintillation probe 71 according to the present modification, biopsy can be performed in a sure manner while sucking the SN 66 along with the surrounding portion.

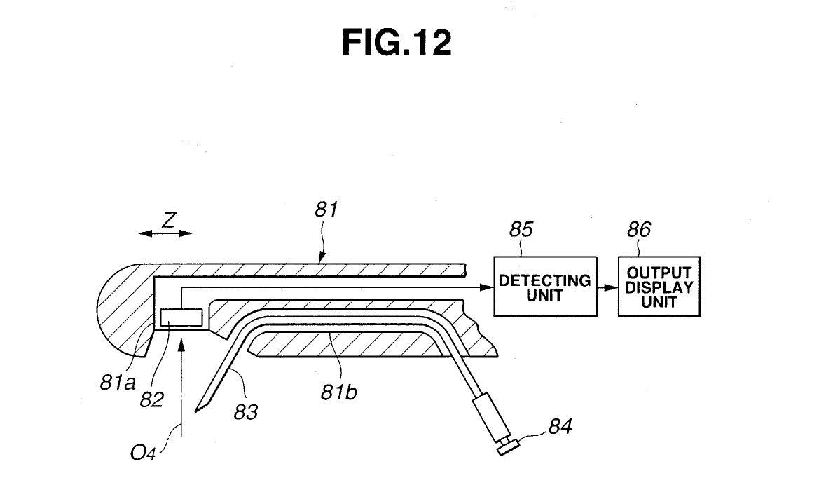

Now, a scintillation probe of a fourth modification corresponding to the scintillation probe 37 applied to the sentinel lymph node observation system 30 according to the above-described second embodiment will be described with reference to FIG. 12.

FIG. 12 is a cross-sectional view illustrating the tip of a scintillation probe according to the present modification.

While the X-ray detecting unit 38 of the scintillation probe 37 according to the above-described second embodiment detects X-rays cast thereon from the front in the probe longitudinal direction Z, an X-ray detecting unit 82 of a scintillation probe 81 according to the present modification can detect X-rays from a direction from the side of the probe longitudinal direction Z. That is to say, the X-ray detecting axis O4 of the X-ray detecting unit 82 is orthogonal to the probe longitudinal direction Z.

As shown in FIG. 12, the scintillation probe 81 of the present modification generally comprises the X-ray detecting unit 82 consisting of an X-ray detector made up of a scintillator, mounted to a side opening unit 81a of the probe, an aspiration biopsy needle 83 which is inserted along an inserting opening 81b of the probe, a puncture needle operating unit 84, a detecting unit 85 for detecting the quantity of visible light obtained by converting X-rays acquired by the X-ray detecting unit 82, and an output display unit 86 for displaying the output of the detecting unit.

In the event of detecting the SN position using the scintillation probe 81 of the present modification, following injecting an RI colloid around the affected portion, while positioning the probe 81 so that the side of the probe 81 faces around the affected portion, the change in the X-ray intensity is observed on the output display unit 86, and the SN position is detected based upon the displayed state. Following detecting of the SN position, a surgeon inserts the aspiration biopsy needle 83 into the SN position so as to take a tissue sample, or place a mark.

With the scintillation probe 81 according to the present modification, SNs at narrow portions which cannot be readily detected by viewing the body cavity from the front can be detected.

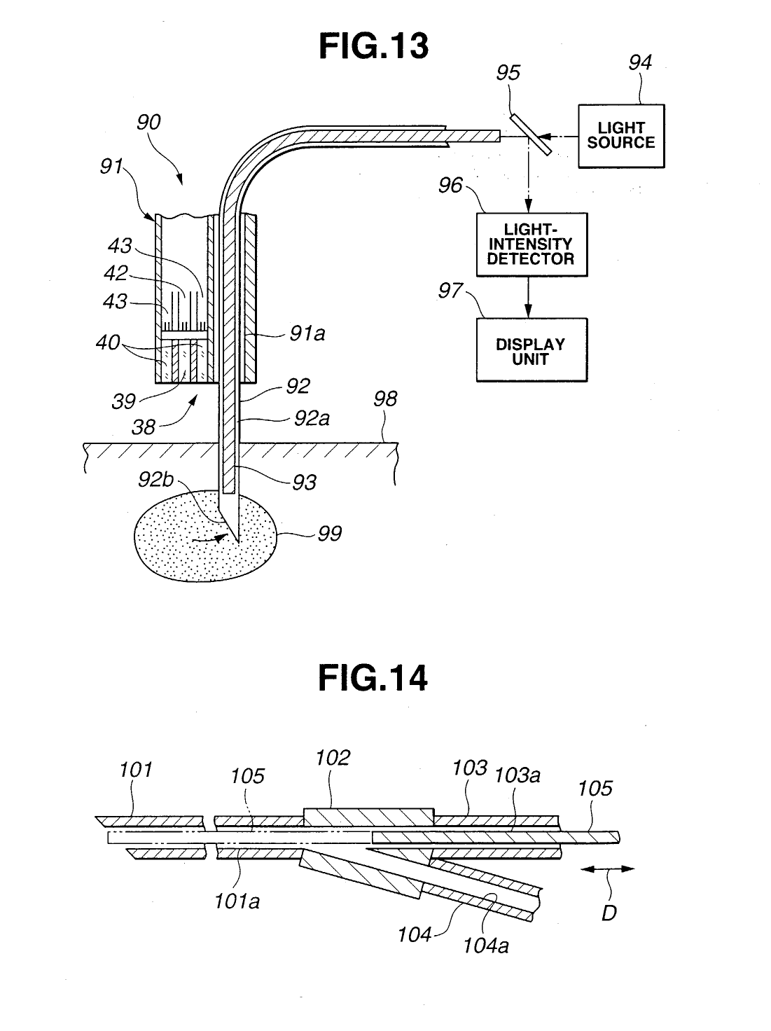

Now, a sentinel lymph node observation system to which the sentinel lymph node detecting method of a third embodiment according to the present invention is applied will be described with reference to FIG. 13.

FIG. 13 illustrates the configuration of a sentinel lymph node observation system and a cross-sectional view of an X-ray detecting unit making up the system according to the present embodiment.

A sentinel lymph node observation system 90 of the present embodiment employs an aspiration biopsy needle wherein an optical fiber is inserted into the inner tube thereof, disposed along the side of the X-ray detecting unit, in the event of taking a tissue sample of the SN with an aspiration biopsy needle following identifying the SN position. Judgment can be made in a sure manner regarding whether or not the portion, into which the puncture needle is inserted, is the SN in which the radioactive substance is accumulated, based upon the information with regard to the change in the light intensity obtained from the optical fiber.

The sentinel lymph node observation system 90 of the present embodiment comprises a scintillation probe 91, the X-ray detecting unit 38 disposed at the tip of the probe 91, an aspiration biopsy needle 92 inserted into an inserting opening 91a on the side of the X-ray detecting unit 38, an optical fiber 93 inserted into an inner tube 92a of the aspiration biopsy needle 92, a half mirror 95 disposed in a tilted position so as to face the optical fiber 93, a light source 94 for generating white light or monochromatic light, a light intensity detecting unit 96, a display unit 97 for displaying output values from the light intensity detecting unit 96, and further an output display unit (not shown) for displaying X-ray information detected by the X-ray detecting unit 38, and so forth.

The X-ray detecting unit 38 comprises the scintillators 39 and 40, optical fibers 42 and 43, and so forth, the same as the X-ray detecting unit (see FIG. 2) applied to the above-described second embodiment.

The optical fiber 93 guides light from the light source 94 to the tip of the aspiration biopsy needle 92, and guides reflected light from the tissue to the base of the endoscope.

The light intensity detecting unit 96 detects the quantity of reflected light obtained by the optical fiber 93.

The SN position can be identified using the sentinel lymph node observation system 90 of the present embodiment having the above-described configuration, with the same method as the observation system 30 according to the above-described second embodiment.

In the event of taking a tissue sample of the SN 99 positioned within the wall of the body cavity 98 using the present observation system 90 following identifying the SN position, light from the light source 94 is guided to the tip of the optical fiber via the half mirror 95. A surgeon inserts the aspiration biopsy needle 92 into the wall of the body cavity 98 in this state. Upon the tip 92b of the aspiration biopsy needle 92 reaching the position of the SN 99, the quantity of reflected light at the tip of the optical fiber 93 markedly changes due to the RI colloid accumulated in the SN 99. The change of reflected light is detected in the light density detecting unit 96 via the half mirror 95, and is displayed on the light density display unit 97. A surgeon can recognize that the aspiration biopsy needle 92 has reached the SN 99 by observing the change of the displayed state.

Note that the light density display unit 97 may notify the user that the aspiration biopsy needle 92 has reached the SN 99 by sound as well as by displaying on the screen.

Following the aspiration biopsy needle 92 reaching the SN 99, the optical fiber 93 is extracted from the inner tube of the aspiration biopsy needle 92, and a tissue sample of the SN 99 can be taken by suctioning.

The sentinel lymph node observation system 90 of the present embodiment has the same advantages as the observation system 30 of the above-described second embodiment, and in particular, in the event of taking the identified SN tissue sample, the sentinel lymph node observation system 90 can detect the depth-wise position of the SN 99 within the wall of the body cavity using the reflect light from the optical fiber 93 in a sure manner.

Now a modification of the aspiration biopsy needle 92 applied to the above-described third embodiment will be described with reference to a cross-sectional view illustrating a puncture needle of the present modification shown in FIG. 14.

With the aspiration biopsy needle 92 applied to the above-described third embodiment, the optical fiber 93 is inserted into the inner tube of the aspiration biopsy needle 92 for specifying the depth of the SN, and it is necessary that the optical fiber 93 be extracted from the inner tube by moving the optical fiber 93 for a long distance when sucking a tissue sample. With the aspiration biopsy needle 101 of the present modification, the movement distance of the optical fiber is reduced, thereby improving operability.

As shown in FIG. 14, with the aspiration biopsy needle 101 according to the present modification, a forked tube 102 is connected to a needle side inner tube portion 101a of the aspiration biopsy needle 101, and the forked tube 102 is branched into a fiber inserting tube 103 and a suction tube 104 on the probe base side. An optical fiber 105 can be inserted into an inner tube portion 103a of the fiber inserting tube 103, and a suction device, which is not shown in drawings, is connected to an inner tube portion 104a of the suction tube 104.

In the event of inserting the aspiration biopsy needle 101 into the SN position within the wall of the body cavity for taking a tissue sampling, the optical fiber 105 is inserted into the tip of the inner tube portion 101a of the aspiration biopsy needle 101. Following inserting the aspiration biopsy needle 101 into the wall of the body cavity and confirming the SN position by the reflection of light from the optical fiber 105, the optical fiber 105 is retracted to the forked tube 102, which is a relatively short distance. The SN tissue sample can be sucked through the inner tube portion 104a of the suction tube 104 and the inner tube portion 101a by operating the suction device with the optical fiber 105 retracted.

With the aspiration biopsy needle 101 of the present modification, it is not necessary that the optical fiber 105 be completely extracted from the aspiration biopsy needle 101 following inserting the aspiration biopsy needle 101 and confirming the SN position, and a tissue sample can be taken with the aspiration biopsy needle 101 by simply moving the optical fiber 105 up to the position of the forked tube 102 for a short distance.

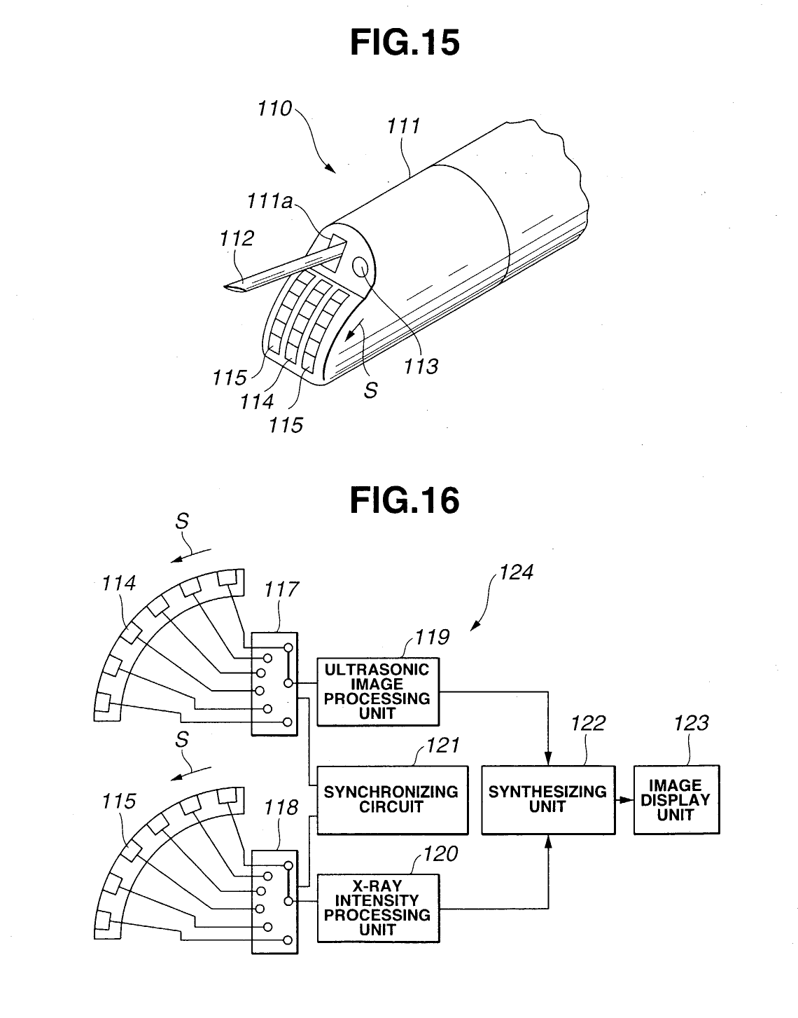

Now, a sentinel lymph node observation system to which a sentinel lymph node observation method of a fourth embodiment according to the present invention is applied will be described with reference to FIGS. 15 through 17.

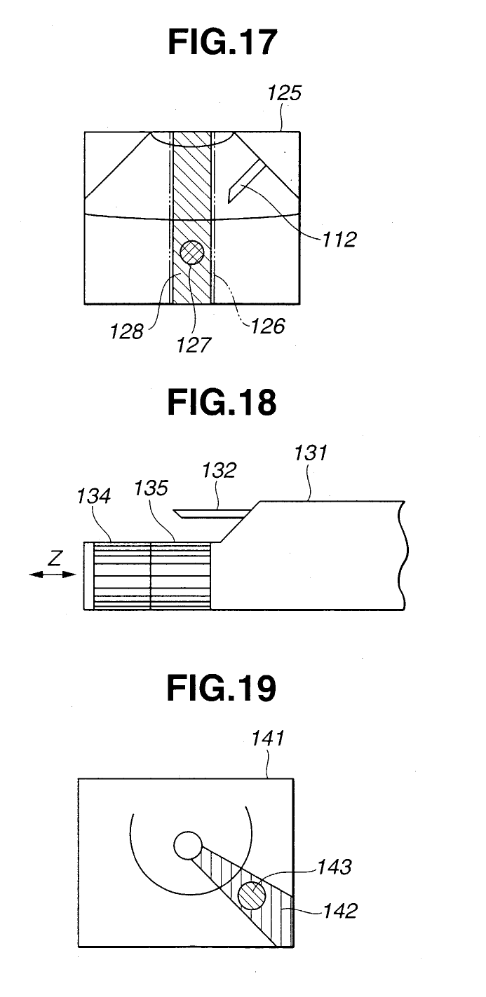

Note that FIG. 15 is a perspective view illustrating the tip of an endoscope inserting portion of a sentinel lymph node observation system according to the present embodiment. FIG. 16 is a block diagram which illustrates an ultrasonic transducer/X-ray detector control unit of the present sentinel lymph node observation system. FIG. 17 illustrates a display screen on which an ultrasonic/X-ray image is displayed in the event of observing the SN position by means of the present sentinel lymph node observation system.

The sentinel lymph node observation system 110 of the present embodiment generally comprises an endoscope which is not shown in drawings, an endoscope inserting portion 111 mounted to the endoscope, an ultrasonic transducer/X-ray detector control unit 124 (see FIG. 16).

As shown in FIG. 15, an injection needle 112 for being inserted into a forceps opening 111a and a visible-light camera unit 113 are disposed at the upper part front part of the tip portion of the inserting portion 111, a convex ultrasonic transducer 114 is disposed at the middle front part of the curved face, and arc-shaped linear-array X-ray detectors 115 are disposed at both sides thereof.

The convex ultrasonic transducer 114 comprises multiple ultrasonic transducer units disposed in an arc shape. In the same way, the linear-array X-ray detector 115 comprises multiple X-ray detector units disposed along an arc face.

As shown in FIG. 16, the ultrasonic/X-ray detecting control unit 124 comprises a switching circuit unit 117, an ultrasonic image processing unit 119 for driving the ultrasonic transducer 114 via the switching circuit unit 117, acquiring echo signals, and performing ultrasonic tomographic image processing, a switching circuit unit 118, an X-ray intensity processing unit 120 for acquiring X-ray signals via the switching circuit unit 118 and performing processing for images indicating the X-ray intensity, a synchronous circuit unit 121 for driving the switching circuit units 117 and 118 in a synchronous manner, a synthesizing circuit unit 122 for synthesizing the output from the ultrasonic image processing unit 119 and the output from the X-ray intensity processing unit 120, and an image display unit 123 for displaying an ultrasonic tomographic image and an X-ray intensity image in a superimposed manner based upon the output from the synthesizing circuit unit 122.

The switching circuit unit 117 sequentially switches the connection with each ultrasonic transducer unit of the ultrasonic transducer 114 in the S direction along the arc. In the same way, the switching circuit unit 118 sequentially switches the connection with each X-ray detector unit of the X-ray detector unit 115 in the S direction along the arc according to the output from the synchronous circuit unit 121.

Now, a method for identifying the SN position using the sentinel lymph node observation system of the present embodiment having the above-described configuration will be described. First of all, an RI colloid, which is a radioactive substance, is injected around the affected portion with the injection needle 112. Following a predetermined time period, the RI colloid flows into the SN position. Following the RI colloid being accumulated in the SN position, the tip of the inserting portion 111 is positioned near the affected position, each transducer unit of the ultrasonic transducer 114 is driven by being sequentially switched in the S direction by the switching circuit unit 117, thereby obtaining ultrasonic tomographic image information near the affected portion in the body. The ultrasonic tomographic image information displays both of the SN lymph nodes and the non-SN lymph nodes.

On the other hand, each X-ray detector unit of the X-ray detector 115 is driven by being sequentially switched in the S direction by the switching circuit unit 118 with the X-ray detector 115 synchronous with the ultrasonic transducer 114, thereby obtaining X-ray intensity information for each X-ray detector unit on the scanning line in front of the inserting portion 111. In the event that the detected X-ray intensity is high, the X-ray intensity display image is displayed as a computer color-enhanced image.

The ultrasonic tomographic image information, and the X-ray intensity image information which is synchronized with the ultrasonic tomographic image information in the S direction scanning are synthesized in the synthesizing circuit unit 122, and the synthesized image is displayed on the image display unit 123.

FIG. 17 illustrates an example of the synthesized image displayed on the image display unit 123. As shown in the drawing, for example, let us say that a lymph node 127 is displayed in a partial ultrasonic tomographic image 126 from a certain transducer unit of the ultrasonic transducer 114 while being driven. In the event that the lymph node 127 is an SN lymph node, the X-ray intensity detected by the corresponding X-ray detector unit of the X-ray detector 115 which is synchronously driven is high. Accordingly, a partial X-ray intensity display image 128 from the X-ray detector unit, which is superimposed on the partial ultrasonic tomographic image 126, is displayed as a computer color-enhanced image, and accordingly, the user can easily recognize that the lymph node 127 is an SN on an ultrasonic tomographic image.

In the event that the lymph node 127 is a non-SN lymph node, the X-ray intensity in the portion is low, so the partial X-ray intensity display image 128 is not displayed as a computer color-enhanced image, and accordingly the user can recognize that the lymph node is not an SN.

Following specifying the SN by the above-described method, a surgeon can take a tissue sample by inserting the aspiration biopsy needle into the portion at which the SN is positioned while observing ultrasonic tomographic images.

With the sentinel lymph node observation system 110 of the present embodiment, the convex ultrasonic transducer 114 and the linear-array X-ray detector 115, which are disposed at the tip of the inserting portion 111, are synchronously driven, ultrasonic tomographic images and X-ray images are synchronously displayed, and a partial X-ray image with high X-ray intensity is displayed as a computer color-enhanced image, so that the user can easily judge whether or not a lymph node displayed on an ultrasonic tomographic image is an SN.

Now, a modification of the inserting portion 111 of the endoscope of the sentinel lymph node observation system 110 according to the fourth embodiment will be described with reference to a side view of an inserting portion in FIG. 18 and a diagram illustrating a display screen on an image display unit shown in FIG. 19.

An inserting portion 131 of the endoscope according to the present modification comprises an injection needle 132 disposed at an upper part of the tip, a visible-light camera which is not shown in the drawing, a linear-array X-ray detector 134 made up of multiple X-ray detector elements, wrapped around the face of the cylinder parallel to the probe longitudinal direction Z of the tip cylinder portion, a linear-array ultrasonic transducer 135 made up of multiple ultrasonic transducer elements, wrapped around the face of the cylinder behind the X-ray detectors 134.

The X-ray detector 134 made up of multiple X-ray detector elements and the ultrasonic transducer 135 made up of multiple ultrasonic transducer elements are synchronously driven in the same way as the above-described fourth embodiment, and ultrasonic tomographic image information, and X-ray image information indicating the X-ray intensity in the same circumferential direction is acquired.

FIG. 19 illustrates an example of a screen 141 on the image display unit which displays the ultrasonic tomographic image and the X-ray image in a superimposed manner. In the event that a lymph node 143 is displayed on the ultrasonic tomographic image and the lymph node is an SN, the X-ray intensity detected in the portion is high, and an X-ray image 142 is displayed as a computer color-enhanced image. Accordingly, the user can easily recognize that the lymph node 143 is an SN.

With the inserting portion 131 of the endoscope according to the present modification, the same advantages as the inserting portion 111 according to the fourth embodiment can be obtained, and in particular, a linear-array ultrasonic transducer 135 made up of multiple ultrasonic transducer elements and a linear-array X-ray detector 134 made up of multiple X-ray detector elements are disposed along the face of the cylinder at the tip of the probe, and accordingly, two-dimensional ultrasonic images and X-ray images for each cross section around the affected portion are simultaneously obtained, and thus the user can identify SNs quickly.

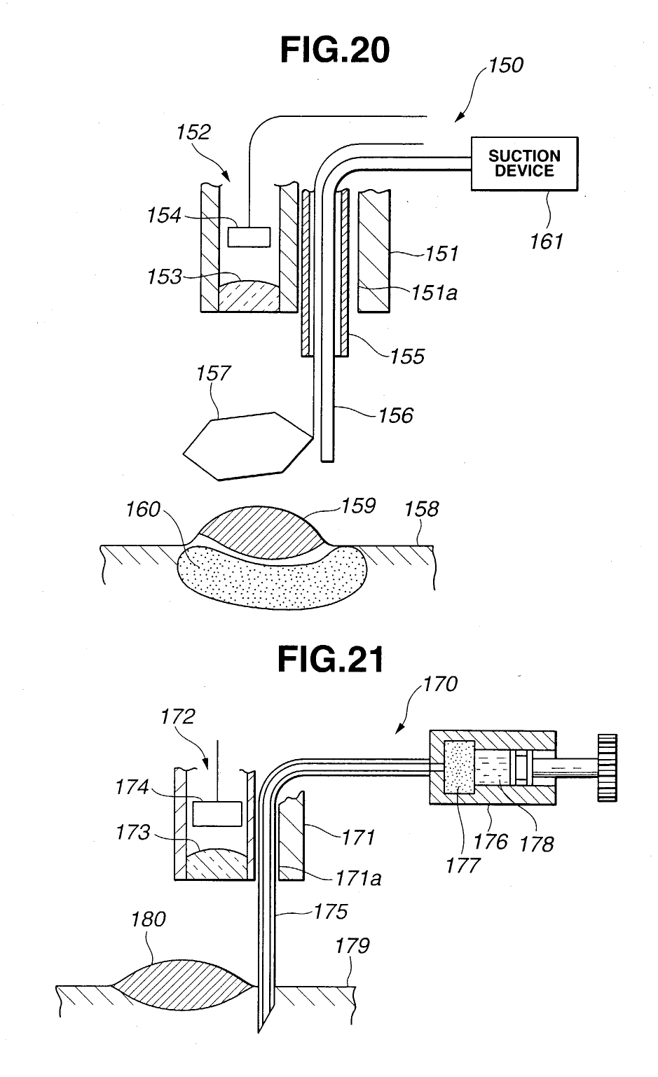

Now, an inserting portion of an endoscope of a sentinel lymph node observation system to which a sentinel lymph node detecting method according to a fifth embodiment of the present invention has been applied will be described with reference to a cross-sectional view of the inserting portion as shown in FIG. 20.

As shown in FIG. 20, an inserting portion 151 of an endoscope of a sentinel lymph node observation system 150 according to the present embodiment comprises an object lens 153 and a CCD 154, making up a body cavity imaging camera unit 152, a surgical tool 155 which is inserted into a forceps opening 151a and a suction device 161.

A suction catheter 156 connected to the suction device 161 and a snare 157 are inserted into the surgical tool 155.

In the event of identifying an SN, a tracer 160 such as an RI colloid is injected around an affected portion 159 of a wall portion in the body cavity 158, but the tracer 160 left around the affected portion 159 interferes with SN detection. Accordingly, with the present sentinel lymph node observation system 150, the affected portion 159 is removed with the snare 157 mounted to the surgical tool 155 of the inserting portion 151, and subsequently, the tracer 160 left around the affected portion 159 is sucked so as to be removed using the suction catheter 156. An SN detecting probe is inserted into the body cavity in this state, and the SN is identified by detecting only the tracer accumulated in the SN.

With the sentinel lymph node observation system 150 of the present embodiment, the tracer 160 left around the affected portion 159 is removed, thereby facilitating detection of SNs, and thus the SN can be identified in a sure manner.

Now, an inserting portion of an endoscope of a sentinel lymph node observation system to which a sentinel lymph node detecting method according to a sixth embodiment of the present invention has been applied will be described with reference to a cross-sectional view of the inserting portion shown in FIG. 21.

As shown in FIG. 21, an inserting portion 171 of an endoscope of a sentinel lymph node observation system 170 according to the present embodiment comprises an object lens 173 and a CCD 174, making up a body cavity portion imaging camera unit 172, an injection needle 175 which is inserted into a forceps opening 171a, and a tracer syringe 176 which is connected to the injection needle 175.

The tracer syringe 176 includes a filter 177 for filtering and removing large colloidal particles contained in a tracer 178 employing an RI colloid or the like.

In the event of identifying the SN by the sentinel lymph node observation system 170 of the present embodiment, the inserting portion 171 of the endoscope is inserted into the body cavity and the injection needle 175 is inserted around an affected portion 180 on the wall of the body cavity 179, the tracer 178 is filtered by the filter 177 of the syringe 176, and is injected around the affected portion 180.

The injected tracer 178 has been subjected to removal of large colloidal particles, and accordingly, situations wherein the lymph node clogs up with the tracer 178 can be avoided, so the tracer flows into the SN, and is accumulated in the SN in a sure manner.

With the sentinel lymph node observation system 170 of the present embodiment described above, in the event of identifying the SN, the injected tracer 178 flows without the lymph node being clogged thereby, so the SN can be identified in a sure manner.

With the first through the sixth embodiments described above, the X-ray detector and the optical imaging device are disposed along the longitudinal direction of the inserting portion so as to observe in the same direction, thereby enabling the SN image being superimposed on the image from the optical imaging device and displaying the superimposed image, and thus enabling providing of a sentinel lymph node detecting method for identifying the SN position in a simple manner.

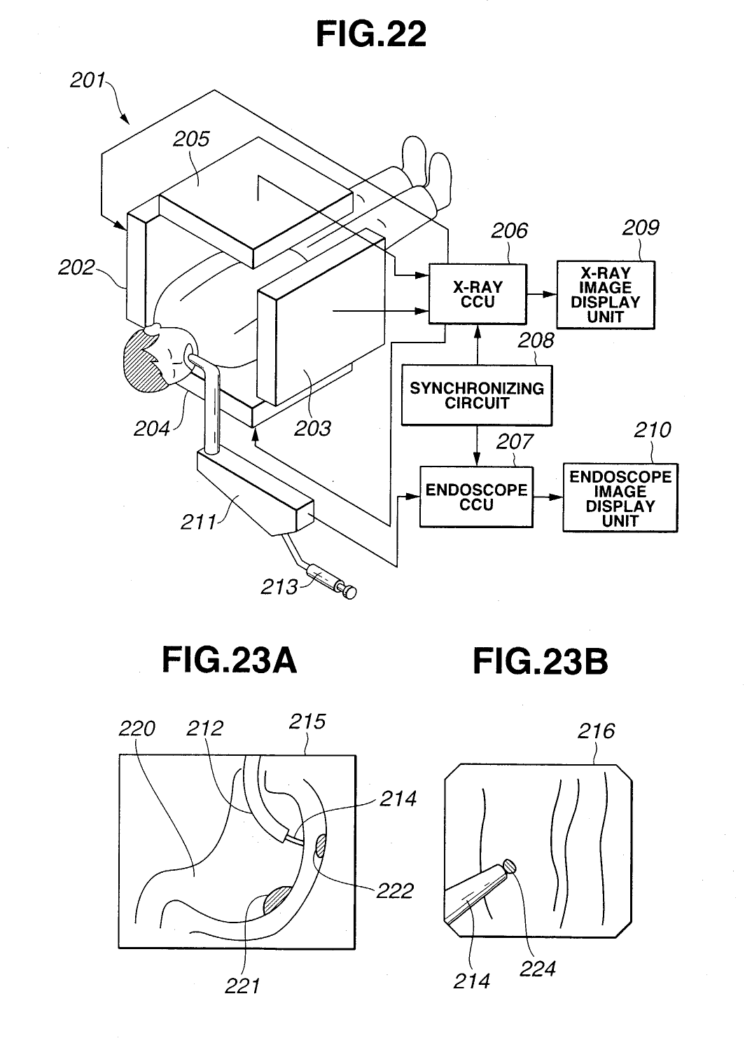

Now, a sentinel lymph node observation system to which a sentinel lymph node observation method according to a seventh embodiment of the present invention has been applied will be described with reference to FIGS. 22 through 23B.

FIG. 22 is a diagram which illustrates the configuration of a sentinel lymph node observation system according to the seventh embodiment. FIG. 23A illustrates a display screen on an X-ray display unit according to the system, and FIG. 23B illustrates a display screen on an endoscope image display unit according to the system.

The sentinel lymph node observation system 201 of the present embodiment generally comprises a flexible endoscope 211, a first X-ray source 202 and a first X-ray camera 203 for generating vertical cross-sectional X-ray images, a second X-ray source 204 and a second X-ray camera 205 for generating horizontal cross-sectional X-ray images, an X-ray CCU (camera control unit) 206 for controlling X-ray cameras for generating X-ray images, an endoscope CCU (camera control unit) 207 for controlling an endoscope camera for generating endoscope images, a synchronizing circuit 208, an X-ray image display unit 209 for displaying a two-dimensional X-ray image by switching between the images from the first and the second X-ray cameras, and an endoscope image display unit 210 for displaying a visible image from the endoscope camera.

The endoscope 211 comprises an inserting portion 212 having a built-in endoscope camera for visible light, which can be inserted into the body cavity (for example, an internal organ such as the stomach), and a marking device 213 which is a marking means.

The marking device 213 includes a dye injector and a marking needle 214 which can be inserted from a forceps opening of the endoscope 211, and marking is made with the marking needle 214 at the corresponding position on the wall of the body cavity underneath which the identified SN is positioned. Note that the marking needle 214 is made of a needle member which absorbs X-rays. Accordingly, the position of the marking needle 214 is displayed on the X-ray image.

The first and second X-ray cameras 203 and 205 detect vertical cross-sectional and horizontal cross-sectional X-ray imaging signals of the body, respectively, that is to say, the first and second X-ray cameras 203 and 205 detect X-rays which have been cast from the X-ray sources 202 and 204, and have not been absorbed by the body. Accordingly, the SN portion in which an X-ray imaging agent is accumulated is detected by the X-ray camera as a portion which does not transmit X-rays.

The X-ray CCU 206 acquires X-ray imaging signals with regard to the vertical and horizontal sections from the first X-ray camera 203 and the second X-ray camera 205, and generates two-dimensional X-ray image information with regard to the vertical and horizontal sections, thereby obtaining three-dimensional information with regard to the body cavity 220.

The synchronizing circuit 208 controls X-ray exposure timing for the X-ray sources 202 and 204 via the X-ray CCU 206, and image output timing for the endoscope CCU 207. That is to say, at the time of X-ray imaging by the X-ray cameras 203 and 205, the endoscope imaging signals are switched to “off”. Conversely, at the time of imaging by the endoscope camera, the X-ray sources 202 and 204 are controlled so as to be “off”.

The sentinel lymph node observation system 201 detects an SN corresponding to an affected portion 221 on the body cavity 220 (see FIG. 23A), and identifies the position of the SN. Prior to the identifying processing, an X-ray imaging agent is injected with an injection needle at around four points deep in the mucous tissue around the affected portion 221. Following the X-ray imaging agent reaching the SN, the examination begins. Note that the X-ray imaging agent is an imaging agent which absorbs X-rays.

In the event of identifying the SN using the sentinel lymph node observation system 201, X-rays are cast from the first and second X-ray sources 202 and 204, vertical cross-sectional and horizontal cross-sectional X-ray images of the body cavity organ of the patient are taken by the first X-ray camera 203 and the second X-ray camera 205, the X-ray CCU 206 outputs X-ray image information, and an X-ray image is displayed on an X-ray image screen 215 of X-ray image display unit 209 as shown in FIG. 23A.

In the event that a surgeon recognizes a portion in which the imaging agent is accumulated in the body cavity 220 which is displayed on the X-ray image screen 215, besides portions near the affected portion 221, judgment is made that the portion is an SN 222 corresponding to the affected portion 221.

In the event that the SN 222 is identified as described above, the tip of the marking needle 214 inserted into the inserting portion 212 of the endoscope 211 is accurately positioned at the wall of the cavity underneath which the SN 222 is positioned as shown in FIG. 23A. Note that positioning of the marking needle 214 is performed while observing both X-ray image screens from the first and the second X-ray cameras 203 and 205.

Visible-light endoscope images from the endoscope camera are acquired by the endoscope CCU 207 and are displayed on the endoscope image display unit 210 with the marking needle 214 being positioned as described above (see FIG. 23B). A surgeon operates the marking device 213 so as to place a mark 224 onto the position on the wall of the body cavity corresponding to the SN 222. Note that in the case of the affected portion 221 shown in FIG. 23A, there is only one SN 222. However, there might be multiple SNs, and in this case, multiple marks are placed onto the wall of the body cavity corresponding to the SNs, respectively.

Identification of the SN ends with the above-described processing. Subsequently, an aspiration biopsy needle is inserted into the endoscope 211, and a surgeon inserts the aspiration biopsy needle into the wall of the cavity from the position of the mark 224 so as to take a tissue sample of the SN 222 while observing endoscope images from the endoscope camera. Judgment is made whether or not the affected portion 221 has spread to the SN based upon the examination results of the tissue sample.

With the examination method using the sentinel lymph node observation system 201 of the present embodiment as described above, the SN can be quickly and accurately identified without laparotomy of the body cavity 220 containing the affected portion 221.

While the sentinel lymph node observation system 201 of the above-described present embodiment employs two sets of an X-ray source and an X-ray camera in two directions for horizontal section and vertical section, it is needless to say that an arrangement may be made wherein only one set of an X-ray source and an X-ray camera in one direction is employed, and the examination is made by rotationally moving the set of the X-ray source and the X-ray camera or rotationally moving the patient.

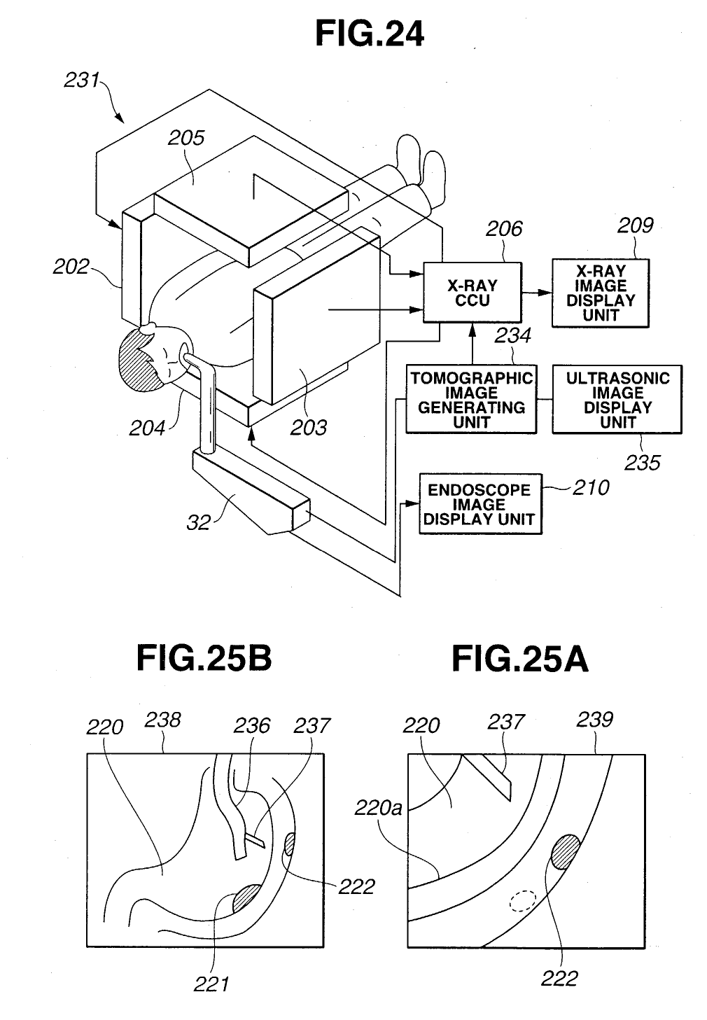

Now, a sentinel lymph node observation system to which a sentinel lymph node observation method according to an eighth embodiment of the present invention has been applied will be described with reference to FIGS. 24 through 25B.

FIG. 24 is a diagram which illustrates the configuration of a sentinel lymph node observation system of the eighth embodiment according to the present invention. FIG. 25A illustrates a display screen on an X-ray display unit according to the system. FIG. 25B illustrates a display screen on an ultrasonic image display unit according to the system.

As shown in FIG. 24, a sentinel lymph node observation system 231 includes an ultrasonic endoscope 232 instead of the endoscope 211 of the system 201 according to the above-described seventh embodiment, and further includes a tomographic image generating unit 234 for ultrasonic images, and an ultrasonic image display unit 235. An endoscope camera (not shown), an ultrasonic transducer (not shown), and an aspiration biopsy needle 237, are mounted to an inserting portion 236 of the ultrasonic endoscope 232. Furthermore, the sentinel lymph node observation system 231 of the present embodiment includes the first X-ray source 202, the first X-ray camera 203, the second X-ray source 204, the second X-ray camera 205, the X-ray CCU 206, the X-ray display unit 209, and the endoscope image display unit 211 for displaying a visible-light image from the endoscope camera in the same way as the seventh embodiment.

Now, procedures for identifying an SN using the sentinel lymph node observation system 231 of the present embodiment will be described. First of all, an X-ray imaging agent is injected around the affected portion 221 in the same way as the system 201 according to the above-described seventh embodiment. Subsequently, X-ray images of the body cavity 220 are taken by the X-ray cameras 203 and 205, and the SN 222 corresponding to the affected portion 221, in which the X-ray imaging agent is accumulated, is displayed on a display screen 238 on the X-ray display unit 209 as shown in FIG. 25A, and is recognized. Note that an X-ray imaging agent which absorbs ultrasonic waves as well as X-rays is employed as the X-ray imaging agent to be injected.

Following detecting of the SN 222, the aspiration biopsy needle 237 of the inserting portion 236, which is inserted into the body cavity 220, is inserted while observing the display screen 238 on the X-ray image display unit 209 displaying images from both of the first and the second X-ray cameras 203 and 205, and the tip thereof is positioned so as to face the wall of the body cavity underneath which the SN 222 is positioned. Note that the aspiration biopsy needle 237 is formed of a material which absorbs X-rays. Accordingly, the user can recognize the inserting position of the aspiration biopsy needle 237 on an X-ray image.

Subsequently, the first and the second X-ray sources are switched to “off”. Tomographic image information containing the SN 222 around the aspiration biopsy needle 237 of the inserting portion 236 is then acquired by the ultrasonic transducer and the tomographic image generating unit 234. The ultrasonic image display unit 235 displays a tomographic image on the display screen 239 as shown in FIG. 25B. A surgeon takes a tissue sample of the SN 222 by inserting the aspiration biopsy needle 237 into the SN 222 underneath the wall of the body cavity and sucking the tissue sample of the SN 222.

With the sentinel lymph node observation system 231 of the present embodiment as described above, marking operations for the identified SN are not required, so biopsy for the SN can be performed quickly. Furthermore, sampling operations for tissue of the SN 222 can be performed while observing ultrasonic tomographic images.

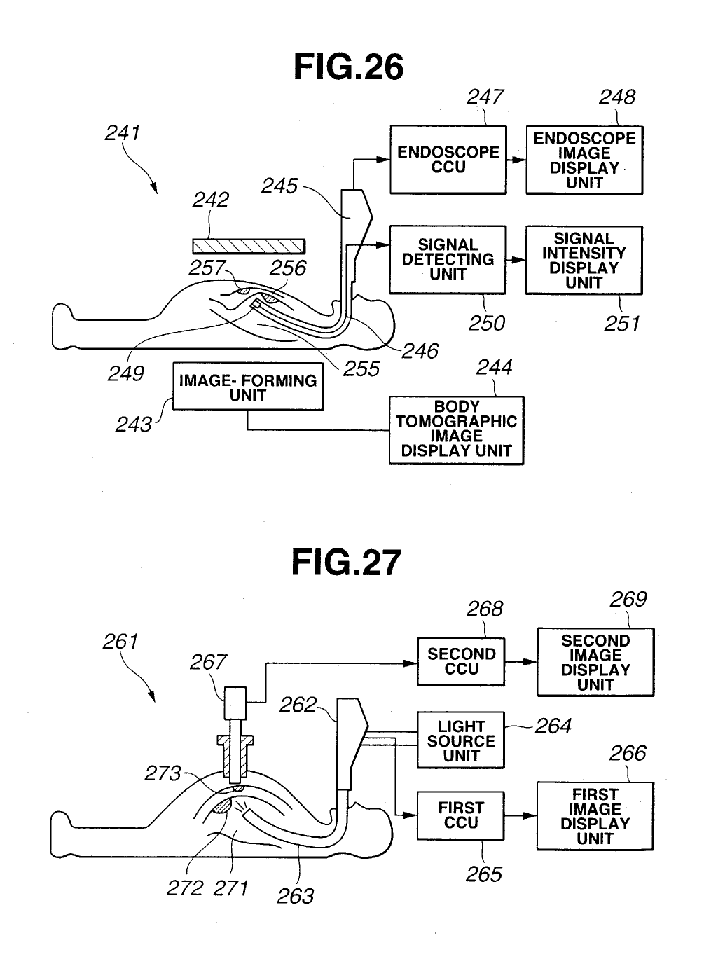

Now, a sentinel lymph node observation system to which a sentinel lymph node detecting method according to a ninth embodiment of the present invention has been applied will be described with reference to a system configuration diagram of the present embodiment shown in FIG. 26.

The present sentinel lymph node observation system 241 is a system which performs identification of an SN using the MRI (magnetic resonance imaging) method, and employs ferrofluid such as Feridex (a registered trademark of an MRI liver imaging agent produced by Eiken Chemical Co., Ltd., generally referred to as “ferumoxides”), as an imaging agent to be injected around the affected portion. Body tomographic image information with regard to the SN around the affected portion is obtained from NMR signals (nuclear magnetic resonance signals) occurring due to the magnetic field of the imaging agent. An antenna portion for detecting the NMR signals is mounted to the probe tip of the inserting portion of the endoscope in order to identify the SN position, the antenna portion is moved so as to detect the SN position, and marking or the like is made so as to identify the SN.

As shown in FIG. 26, the present system 241 generally comprises a magnet 242 which is a magnetic field generating means for generating a magnetic field from the outside of the body in order to obtain two-dimensional or three-dimensional images, a imaging unit 243 which is an imaging means for detecting the nuclear magnetic resonance state occurring due to the magnetic field and generating an MRI image, a body tomographic image display unit 244 for displaying a body tomographic image, an endoscope 245, a probe 246 which is a probe means which is inserted to a forceps opening of the endoscope 245, a coil-shaped antenna 249 which is disposed at the tip of the probe 246, a signal detecting unit 250 for acquiring NMR signals from the antenna 249, a signal intensity display unit 251 for displaying the output intensity from the signal detecting unit 250 by image or sound, an endoscope CCU 247, and an endoscope image display unit 248.

The body tomographic image display unit 244 is a display unit for displaying a two-dimensional or three-dimensional image from MRI imaging signals obtained by the imaging unit 243.

The probe 246 is a probe with a small diameter, which includes a built-in antenna 249 and can be inserted into the body cavity 255 in order to observe the body cavity 255.

The antenna 249 is a coil-shaped antenna for detecting NMR signals occurring due to the magnetic field, which is positioned at the tip of the probe 246.

Now, procedures for identifying an SN using the sentinel lymph node observation system 241 of the present embodiment will be described. First of all, ferrofluid is injected around an affected portion 256 as an imaging agent so that the imaging agent is accumulated in an SN 257 corresponding to the affected portion 256 under observation by the endoscope 245. An image indicating the position of the SN 257 is displayed on the body tomographic image display unit 244 by the imaging unit 243.

Subsequently, the tip of the probe 246 of the endoscope 245 is moved within the body cavity 255 while observing images displayed on the endoscope image display unit 248 as well as images displayed on the body tomographic image display unit 244, and positioning of the tip of the probe 246 is made at the position wherein NMR signals from the antenna 249 are maximal. At this time, the tip of the probe 246 points to the position of the wall of the body cavity underneath which the SN 257 is positioned.

Marking is then made onto the wall of the body cavity to which the tip of the probe 246 points, with a marking needle, so that the SN position is identified. Subsequently, a surgeon takes a tissue sample of the SN 257 by inserting a biopsy needle into the marked portion on the wall of the body cavity while observing images displayed on the endoscope image display unit 248 or tomographic images by the MRI.

Note that an arrangement may be made wherein, following identifying the position of the SN 257 using the antenna 249 of the probe 246, a tissue sample of the SN 257 is taken by inserting a biopsy needle without marking while observing an ultrasonic tomographic screen using an ultrasonic endoscope. Or, the tissue sampling may be performed following marking.

The sentinel lymph node observation system 241 of the present embodiment requires no special X-ray equipment. Furthermore, high-speed MRI image processing also enables biopsy to be performed while observing displayed MRI images in real time, thereby providing an observation system with great ease-of-use.

Now, a sentinel lymph node observation system 261 to which a sentinel lymph node detecting method according to a tenth embodiment of the present invention has been applied will be described with reference to a system configuration diagram of the present embodiment shown in FIG. 27.

With the present sentinel lymph node observation system 261, in the event of identifying an SN position corresponding an affected portion, a dye-type imaging agent is employed as a tracer, near-infrared light is cast from the body cavity on the SN in which the dye-type imaging agent is accumulated, so that the SN position is identified by a camera of a peritoneoscope inserted from the outside of the body.

As shown in FIG. 27, the sentinel lymph node observation system 261 of the present embodiment generally comprises a first endoscope 262 having an inserting portion 263 for being inserted into the body cavity 271, a light source unit 264 for the endoscope, a first CCU 265 for controlling of imaging of a camera built in the endoscope, a first image display unit 266 for displaying images from the endoscope camera, a peritoneoscope 267 of a second endoscope which is inserted under partial laparotomy of the abdominal cavity of a patient, a second CCU 268 for controlling of imaging of a camera build in the peritoneoscope, and a second image display unit 269 for displaying images from the camera build in the peritoneoscope.

The present observation system employs an imaging agent which absorbs light in the range of visible light through infrared light, e.g., ICG, as a dye-type imaging agent for identifying an SN. The dye-type imaging agent is injected around an affected portion 272 from the side of the body cavity 271 with an injecting needle of the endoscope 262 while observing endoscope images.

The light source unit 264 is a light source unit which emits rays in the range of visible light through near-infrared light, in particular, emits near-infrared light which can readily penetrate a body. The near-infrared light is cast on the wall of the body cavity from the tip of the inserting portion 263 of the endoscope 262.

The camera build in the tip of the peritoneoscope 267 is a near-infrared-light camera which can take images from near-infrared light.

Now, procedures for identifying the position of the SN 273 corresponding to the affected portion 272 using the sentinel lymph node observation system 261 of the present embodiment will be described. First of all, a patient is subjected to partial laparotomy of the abdominal cavity, and the peritoneoscope 267 is inserted thereinto. The abdominal cavity is kept inflated with CO₂ gas. While observing the display screen of the first image display unit 266 from the side of the body cavity, ICG, which is employed as an imaging agent, is injected around the affected portion 272 with the injecting needle of the endoscope 262. Near-infrared light is cast on the wall of the body cavity 271 from the tip of the inserting portion 263 of the endoscope 262.

The near-infrared light penetrates the inner wall and the outer wall of the body cavity 271, and is cast on the camera mounted to the tip of the peritoneoscope 267. In the event that the SN 273, in which the ICG is accumulated, is positioned on the outer wall of the internal organ in front of the camera of the peritoneoscope 267, the near-infrared light is absorbed in the SN 273, so the shadow thereof is captured by the peritoneoscope, which is displayed on the second image display unit 269. Note that the SN position is searched while changing the tilt of the peritoneoscope 267.

A surgeon can take a tissue sample corresponding to the SN, which has been detected by the peritoneoscope 267, by inserting a biopsy needle from the side of the peritoneoscope 267 or the side of the endoscope 262.

With the above-described sentinel lymph node observation system 261, in the event of identifying the SN position with near-infrared light which is cast from the side of the body cavity, even if the body internal organ is covered with fat the near-infrared light penetrates the fat, and accordingly, the image of the SN can be captured in a sure manner.

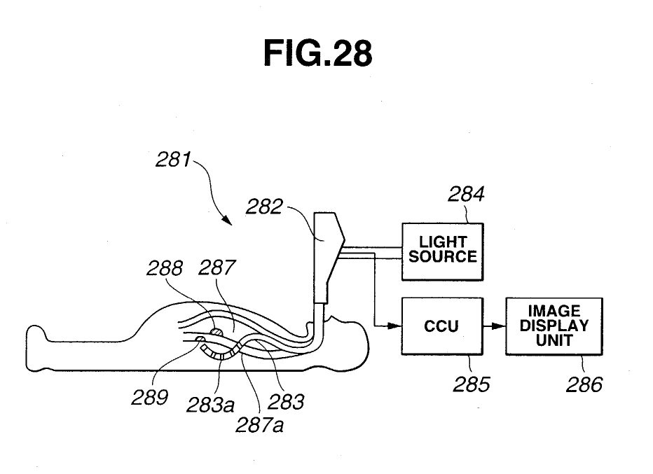

Now, a sentinel lymph node observation system 281 to which a sentinel lymph node detecting method according to a eleventh embodiment of the present invention has been applied will be described with reference to a system configuration diagram of the present embodiment shown in FIG. 28.

With the sentinel lymph node observation system 281 of the present embodiment, an inserting portion of an endoscope is inserted into the side of the abdominal cavity external of the internal organ through an opening provided on the body internal organ cavity. The SN position is identified based upon observed image information with regard to the abdominal cavity.

As shown in FIG. 28, the sentinel lymph node observation system 281 of the present embodiment generally comprises an endoscope 282 having an inserting portion 283, an observing camera built in the tip of the inserting portion 283, an endoscope light source unit 284, an observing camera CCU 285, and an image display unit 286 for displaying endoscope observed images.

The inserting portion 283 of the endoscope 282 has multiple bending portions 283a along the tip thereof.

Now, procedures for identifying an SN position corresponding to an affected portion using the sentinel lymph node observation system 281 of the present embodiment having the above-described configuration will be described. First of all, a through hole 287a to the abdominal cavity is provided on the wall of the body internal organ cavity 287 by means of an incision tool of the endoscope 282. ICG, which is a dye-type imaging agent is injected at four points around an affected portion 288 of the body cavity 287 so that the ICG is accumulated in an SN 289.

The inserting portion 283 of the endoscope 282 is inserted into the through hole 287a on the wall of the body cavity 287 in the state that the injected ICG is accumulated. The dyed SN 289 is displayed on the image display unit 286 by observing the outer wall of the internal organ from the side of the abdominal cavity while bending the inserting portion 283 on the bending portions 283a.

Following the SN 289 being displayed on the display screen of the image display unit 286 and the position thereof being identified, a surgeon can take a tissue sample of the SN 289 by inserting an aspiration biopsy needle into the inserting portion 283.

With the sentinel lymph node observation system 281 of the present embodiment, the SN 289 is observed from the outer wall of the internal organ, and thus the SN 289 which is generally positioned along the outer wall of the internal organ can be accurately identified.

What is claimed is:

1. A sentinel lymph node detecting method by a sentinel lymph node observation system, the system comprising:
an inserting portion with a small diameter which can be inserted into the body cavity;
an X-ray detector at the tip of the inserting portion for two-dimensionally detecting X-rays; and
an optical imaging device for taking visible-light images;
wherein the X-ray detector and the optical imaging device are positioned at the inserting portion in the longitudinal direction thereof, and are closely positioned one to another so as to observe in the same direction.

2. A sentinel lymph node detecting method according to claim 1, wherein the range over which the X-ray detector observes is less than the range over which the optical imaging device observes.

3. A sentinel lymph node detecting method according to claim 1, wherein a two-dimensional image from the X-ray detector is displayed on an image of the body cavity from the optical imaging device, in a superimposed manner.

4. A sentinel lymph node detecting method according to claim 3, wherein portions with high radiation density in a two-dimensional image from the X-ray detector are displayed as a computer color-enhanced image.

5. A sentinel lymph node detecting method according to claim 1, wherein the X-ray detector forms a curved face, and has directivity for detection in the direction orthogonal to the curved face.

6. A sentinel lymph node detecting method according to claim 5, wherein a layer having a plurality of collimator means for yielding directivity is disposed in front of the light-receiving face in the X-ray detector.

7. A sentinel lymph node detecting method by a sentinel lymph node observation system, the system comprising:
an inserting portion with a small diameter which can be inserted into a forceps opening of an endoscope; and
an X-ray detector disposed at the tip of the inserting portion for detecting X-rays;
wherein the X-ray detector is separated into an inner detecting unit and an outer detecting unit, and has a detecting means for detecting the differential between the X-ray intensities detected by the detecting portions.

8. A sentinel lymph node detecting method according to claim 7, wherein the outer detecting portion is made up of a plurality of detecting elements which generally cover the inner detecting portion.

9. A sentinel lymph node detecting method using a sentinel lymph node observation system, the system comprising:
an inserting portion with a small diameter which can be inserted into the body cavity;
an X-ray detector disposed at the tip of the inserting portion; and
an ultrasonic detector disposed at the tip of the inserting portion;
wherein the X-ray detector consists of a linear-array X-ray detector, and the ultrasonic transducer consists of a convex ultrasonic transducer;
and wherein the linear-array X-ray detector and the convex ultrasonic transducer are synchronously driven in the same direction by scanning;
and wherein tomographic image information obtained from the convex ultrasonic transducer and X-ray intensity image information detected by the liner-array X-ray detector is displayed in a synchronous manner.

10. A sentinel lymph node detecting method according to claim 9, wherein X-ray intensity image information detected by the liner-array X-ray detector is displayed as a computer color-enhanced image in the event that the detected X-ray intensity is high.

11. A sentinel lymph node detecting method using a sentinel lymph node observation system, the system comprising:
an inserting portion with a small diameter which can be inserted into the body cavity,
an ultrasonic detector disposed at the tip of the inserting portion, and
an X-ray detector disposed at the tip of the inserting portion;
wherein the ultrasonic transducer consists of a linear-array ultrasonic transducer made up of a plurality of ultrasonic transducer elements, wrapped around the cylinder face with the same axis as the cylinder face of the tip of the inserting portion,
and wherein the X-ray detector consists of a linear-array X-ray detector made up of a plurality of X-ray detector elements, wrapped around the cylinder face of the tip of the inserting portion,
and wherein the liner-array ultrasonic transducer and the liner-array X-ray detector are synchronously driven in the same direction,
and wherein tomographic image information obtained from the liner-array ultrasonic transducer and X-ray intensity image information detected by the liner-array X-ray detector is displayed in a synchronous manner.

12. A sentinel lymph node detecting method according to claim 11, wherein X-ray intensity image information detected by the liner-array X-ray detector is displayed as a computer color-enhanced image in the event that the detected X-ray intensity is high.

13. A sentinel lymph node detecting method comprising:
an X-ray source and an X-ray camera, for observing sentinel lymph nodes near affected portions in which an imaging agent for absorbing X-rays has been accumulated beforehand,
an endoscope for observing the body cavity, and marking means inserted from a forceps opening of the endoscope;
wherein marking is made with the marking means onto the inner wall of the body cavity organ near the portion in which the imaging agent has been accumulated.

14. A sentinel lymph node detecting method according to claim 13, wherein image signals for the X-ray camera and image signals for the endoscope are synchronous, so that at the time of taking of X-ray images, image signals for the endoscope are switched to “off”.

15. A sentinel lymph node detecting method according to claim 13, wherein the marking means is made up of a material which absorbs X-rays.

16. A sentinel lymph node detecting method which uses:
magnetic field generating means for imaging sentinel lymph nodes near affected portions in which ferrofluid has been accumulated beforehand,
imaging means for imaging nuclear magnetic resonance occurring due to the magnetic field, and
probe means with a small diameter which can be inserted into the body cavity;
wherein an antenna is disposed at the tip of the probe means for detecting signals occurring due to the magnetic field.

17. A sentinel lymph node detecting method according to claim 16, wherein the antenna is a coil for detecting NMR signals.

18. A sentinel lymph node detecting method which uses:
a first endoscope for lighting up the body cavity containing affected portions around which a substance has been injected for imaging sentinel lymph nodes beforehand, and
a second endoscope for observing transmitted light from the first endoscope, from the abdominal cavity side.

19. A sentinel lymph node detecting method according to claim 18, wherein the second endoscope is a peritoneoscope.

20. A sentinel lymph node detecting method according to claim 18, wherein the substance absorbs light in the range of visible light through infrared light.

21. A sentinel lymph node detecting method according to claim 18, wherein the substance is ICG.

22. A sentinel lymph node detecting method which uses:
an X-ray source and an X-ray camera for observing sentinel lymph nodes near affected portions in which an imaging agent for absorbing X-rays and ultrasonic waves has been accumulated beforehand,
an endoscope comprising
an imaging device for observing the body cavity and
an ultrasonic transducer for imaging tomographic images of tissue; and
needle means inserted from a forceps opening of the endoscope;
wherein a tissue sample is taken of the portion in which the imaging agent has been accumulated.

23. A sentinel lymph node detecting method according to claim 22, wherein the tip of the needle means is made up of a material which absorbs X-rays.
